(19) United States
(12) Patent Application Publication
(10) Pub. No.: US 2025/0173873 A1
(43) Pub. Date: May 29, 2025

(54) SYSTEMS AND METHODS FOR IMAGE NAVIGATION USING ON-DEMAND DEEP LEARNING BASED SEGMENTATION

(71) Applicant: Digital Surgery Systems, Inc., Goleta, CA (US)

(72) Inventors: Mannat Rana, Goleta, CA (US); Kyle Kam, Goleta, CA (US); Murat Karaorman, Goleta, CA (US)

(21) Appl. No.: 18/962,340

(22) Filed: Nov. 27, 2024

Related U.S. Application Data
(60) Provisional application No. 63/602,967, filed on Nov. 27, 2023.

(51) Int. Cl. G06T 7/11; A61B 34/00; A61B 34/30; B25J 9/14; G06T 7/136; G06T 11/00

(52) U.S. Cl. CPC G06T 7/11; A61B 34/25; A61B 34/30; B25J 9/14; G06T 7/136; G06T 11/00; A61B 2034/254; G06T 2207/20081; G06T 2207/20104; G06T 2207/20116; G06T 2207/20152; G06T 2207/30004

(57) ABSTRACT

A computing system and method are provided for image navigation using on-demand deep learning based segmentation. An example system may comprise an exoscope configured to capture image data from a field of view; an image segmentation module; an intent recognition module to capture a user's intent; and one or more robotic arms configured to move the exoscope. The system may receive, via the exoscope, image data relating to an image or a video stream of a surgical site; generate, via the image segmentation module, an augmented image comprising a plurality of labeled regions overlaying the surgical site; receive, via the intent recognition module, a voice command selecting a labeled region of the plurality of labeled regions; and cause, via the one or more robotic arms, a movement of the exoscope so that the selected labeled region is within the field of view of the exoscope after the movement.

### PRIORITY CLAIM

This patent application claims priority to U.S. Provisional Patent Application No. 63/602,967, filed on Nov. 27, 2023, the entireties of which are hereby incorporated by reference and relied upon.

### BACKGROUND

An exoscope is a high-definition digital imaging system that enables surgeons to see a magnified, three-dimensional (3D) image of a surgical area via a display. A surgeon typically relies on the exoscope during surgeries or microsurgeries involving higher degree of detail, such as procedures on the brain, eyes and spinal cord. The exoscope may be positioned to capture image and/or video data from the surgical data, for example, through one or more robotic arms. The robotic arms may be used to move the exoscope, for example, to adjust the field of view of the exoscope. Furthermore, movement of the exoscope may be executed by a surgeon's command or input. For example, a surgeon may rely on hand controls (e.g., one or more handles of the exoscope) or a voice command (e.g., via simple commands such as “Move Right” or “Move East” entered into a microscope) to move the exoscope, which may be attached to the end of a robotic arm of a surgical navigation system.

However, there is a desire and need for developing more precise systems and methods for navigating and positioning the exoscope. For example, although the surgeon may rely on voice commands (e.g., such as “Move Right” or “Move East) and hand controls to move the exoscope in the direction desired by the surgeon, such surgeon-driven commands typically fail to provide the higher degree of precision required for positioning the exoscope to accurately face a target location. The movement of the exoscope (e.g., via robotic arms) may cause the exoscope's field of view to easily cross over the location of a surgical area that is desired to be captured, or the field of view may not easily center at the desired location. Additionally, the need to position the exoscope toward a desired location results in frequent interruptions of the surgery, particularly as surgical areas change through the course of a surgery. The interruptions may often impact the performance of a surgery, by causing delays, diverting a surgeon's attention, or otherwise increasing the risk of mistakes.

Various embodiments of the present disclosure address one or more shortcomings of conventional couplers described above.

### SUMMARY

The present disclosure provides new and innovative systems and methods for image navigation using on-demand deep learning based segmentation.

In one embodiment, an example system may comprise an exoscope configured to capture image data from a field of view; an image segmentation module; an intent recognition module to capture a user's intent; one or more robotic arms configured to move the exoscope; a processor; and memory. The memory stores computer-executable instructions that, when executed by the processor, causes the system to: receive, via the exoscope, image data relating to an image or a video stream of a surgical site; generate, via the image segmentation module, an augmented image comprising a plurality of labeled regions overlaying the surgical site; receive, via the voice intent recognition module, a voice command selecting a labeled region of the plurality of labeled regions; and cause, via the one or more robotic arms, a movement of the exoscope so that the selected labeled region is within the field of view of the exoscope after the movement.

In an embodiment, the plurality of labeled regions comprise one or both of: a plurality of color coded regions; or a plurality of textually labeled regions.

In an embodiment, the one or more robotic arms include at least one of a pneumatic arm and a hydraulic arm.

In an embodiment, the system further comprises an instrument control module. The instrument control module may be configured to receive a command signal caused by a tapping gesture on a surgical instrument while the surgical instrument is pointed towards a labeled region of the plurality of labeled region.

In an embodiment, the instructions, when executed by the processor, further cause the system to: receive, via the instrument control module, an instrument command selecting a second labeled region of the plurality of labeled regions; and cause, via the one or more robotic arms, a second movement of the exoscope so that the second selected labeled region is within the field of view of the exoscope.

In an embodiment, the instructions, when executed by the processor, cause the system to generate the augmented image by: segmenting, using a deep learning model, the image of the surgical site into a plurality of regions.

In an embodiment, the system further comprises a display configured to output the augmented image.

In an additional embodiment, the instructions, when executed by the processor, may further cause the system to: train the deep learning model using training data comprising a plurality of reference image data having a plurality of recognized regions associated with the reference image data, and employ deep learning methods to associate each pixel of the image with a corresponding anatomical structure.

Moreover, the instructions, when executed by the processor, may cause the system to segment, using the deep learning model, the image of the surgical site into the plurality of regions by at least one of: clustering regions of the image of the surgical site based at least upon one of threshold intensity values for pixels; using seed points of the image for growing regions based on similarity criteria; and applying edge detection, watershed segmentation, or active contour detection.

In an example, a method for image navigation using on-demand deep learning based segmentation is disclosed. The method comprises: receiving, by a computing system having one or more processors, via an exoscope, image data for an image of a surgical site; generating, via a segmentation module associated with the computing system, an augmented image comprising a plurality of labeled regions overlaying the surgical site; displaying the augmented image; receiving, by the computing system, a user input selecting a labeled region of the plurality of labeled regions; and causing, via one or more robotic arms supporting the exoscope, movement of the exoscope so that the selected labeled region is within a field of view of the exoscope after the movement.

In an embodiment, the user input is one or both of: a voice command selecting a labeled region of the plurality of labeled regions; or a tapping gesture on a surgical instrument while the surgical instrument is pointed towards a labeled region of the plurality of labeled regions.

In an embodiment, the method may further comprise, prior to generating the augmented image: receiving a first user input to initiate segmentation. The augmented image may be generated responsive to the first user input.

In an example, computer-executable methods are disclosed to describing one or more steps, methods, or processes described herein.

In an example, a non-transitory computer-readable medium for use on a computer system is disclosed. The non-transitory computer-readable medium may contain computer-executable programming instructions may cause processors to perform one or more steps or methods described herein.

### BRIEF DESCRIPTION OF THE FIGURES

FIG. 1 shows a diagram of a surgical environment, which includes an exoscope and a surgical computing system, for performing on-demand deep learning based segmentation, according to an example embodiment of the present disclosure.

FIG. 2 is a block diagram of an example surgical computing system for performing on-demand deep learning based segmentation, in accordance with a non-limiting embodiment of the present disclosure.

FIG. 3 is a block diagram of an example method for performing on-demand deep learning based segmentation, in accordance with a non-limiting embodiment of the present disclosure.

FIGS. 4, 5, and 6 are image illustrations showing on-demand deep learning based segmentation, in accordance with a non-limiting embodiment of the present disclosure.

### DETAILED DESCRIPTION

As previously discussed, there is a desire and need for developing more precise systems and methods for navigating and positioning the exoscope, particularly since the exoscope is used for surgeries or microsurgeries involving higher degree of detail (e.g., procedures on the brain, eyes and spinal cord). Conventional voice command and hand control systems for moving the exoscope in the direction desired by the surgeon typically fail to provide the higher degree of precision required for positioning the exoscope to accurately and/or precisely face a target location. Additionally, the need to position the exoscope toward a desired location results in frequent interruptions of the surgery, impacting the performance of a surgery, by causing delays, diverting a surgeon's attention, or otherwise increasing the risk of mistakes.

The present disclosure provides new and innovative systems and methods to address and overcome the above described issues. For example, in at least one embodiment, the present disclosure describes a closed loop system and method based on the capture and identification of specific image landmarks in the field of view of the exoscope. The present disclosure describes using deep learning based image segmentation techniques to segment image data captured by the exoscope. Based on the segmentation, the present disclosure describes systems and methods for generating labels (e.g., color and/or text) and overlaying the segmented regions with those labels, to generate an augmented image and/or video stream. Various embodiments of the present disclosure further describe enabling a surgeon to select an identified labeled region (e.g., color-coded and/or labeled by text). Various embodiments further describe the use of that selected labeled region as a landmark location to generate a command signal. The command signal may cause the exoscope to move (e.g., via signals to robotic arms supporting the exoscope), so that the field of view of the exoscope is centered and/or refocused on the selected labeled region after the movement. For example, the surgeon can command the exoscope to move to a specific location by saying “Go to [textually labeled] region” and/or “Go to blue region” where “[textually labeled]” is shorthand for the specific identifier of the region indicated by the labeled text overlaying the region on the augmented image. Also or alternatively, the surgeon can command the exoscope to move to the specific location via a tapping gesture on a surgical instrument used by the surgeon while the surgical instrument is pointed towards the labeled region corresponding to the specific location.

### Exemplary Surgical Environment

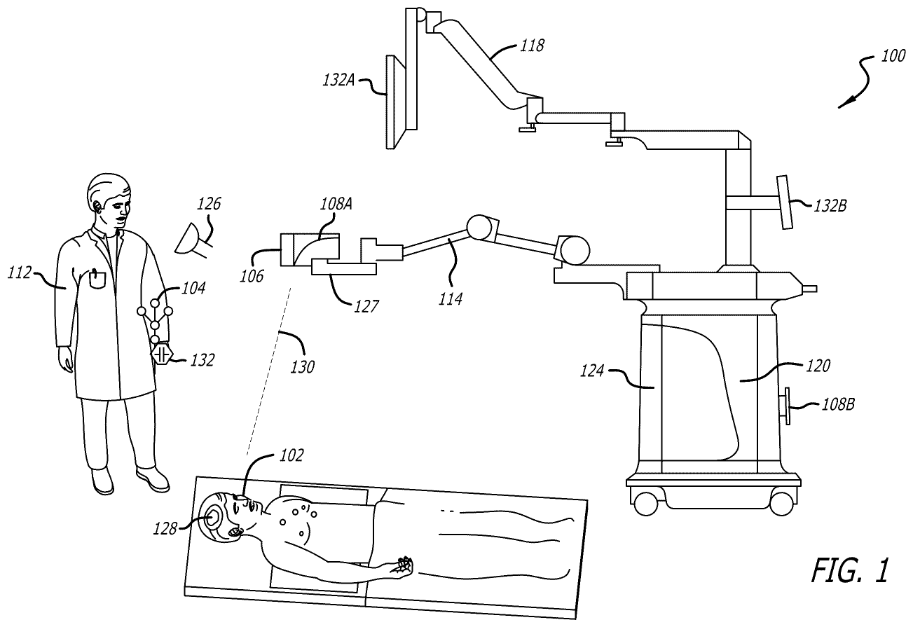

FIG. 1 shows a diagram of a surgical environment 100 including an exoscope 106 and a surgical computing system 120 for performing on-demand deep learning based segmentation, according to an example embodiment of the present disclosure.

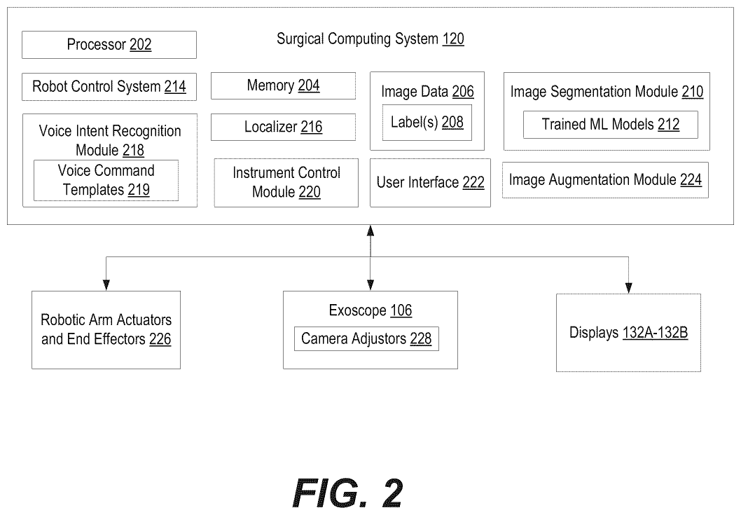

The surgical environment 100 may include: a patient 102 that is subjected to, or intended to be subjected to, a surgical or microsurgical procedure; surgical instruments or tools 104; an exoscope 106 (also referred to herein as “exoscope camera” or “camera” for simplicity); a computing system 120 configured for performing image navigation using deep learning based segmentation described herein (also referred to herein as “surgical computing system” or “computing device”); one or more robotic controls (e.g., hand controls 108A along exoscope 106 or foot pedals 108B); one or more robotic arms 114 (including any pneumatic arms or hydraulic arms 118 and 127); a surgical cart 124; a surgeon 112, one or more microphones 126 (e.g., for facilitating voice commands), and one or more displays 132A-132B. The computing system 120, including one or more example components thereof, is further described in relation to FIG. 2.

The computing system 120 may enable the surgeon 112 to automatically direct a field of view 130 of the exoscope 106 towards a desired location in a surgical site 128 of the patient 120. The surgeon 112 may view, via one or more displays 132A-132B, an image and/or video stream showing labeled regions of the field of view 130 of the exoscope 106. The image and/or video stream may be based on image data captured by the exoscope 106 and may be augmented by the computing system 120 based on systems and methods described herein. The surgeon may select one or more of the labeled regions of the field of view 130. The selection may cause the exoscope 106 to move, for example, via movement of one or more robotic arms 114 and 127 to allow the field of view 130 to be centered at the selected labeled region after the movement. The surgeon 112 may cause the selection using a voice command (e.g., verbally indicating the labeled region) that is detected and received via one or more microphones 126. Also or alternatively, the surgeon may cause the selection by pointing the surgical instrument 104 towards the labeled region that the surgeon intends to select, and then tapping the surgical instrument 104. In some embodiments, the tap may be detected via a tap sensor 132 associated with the surgical instrument 104.

### Example Surgical Computing System

FIG. 2 is a block diagram of an example surgical computing system 120 for performing on-demand deep learning based segmentation, in accordance with a non-limiting embodiment of the present disclosure. As shown in FIG. 2, the example surgical computing system 120 may include but are not limited to: a processor 202; a memory 204; image data 206 (e.g., stored in memory 204); an image segmentation module 210; a robot control system 214; a localizer 216; a voice intent recognition module 218; an instrument control module 220; a user interface 22; and an augmentation module 224. The surgical computing system 120 may further comprise, may be communicatively linked to, and/or may otherwise be associated with one or more robotic arm actuators and end effectors 226, the exoscope 106, and the displays 132A-132B.

The processor 202 may comprise any one or more types of digital circuit configured to perform operations on a data stream, including functions described in the present disclosure. The memory 204 may comprise any type of long term, short term, volatile, nonvolatile, or other memory and is not to be limited to any particular type of memory or number of memories, or type of media upon which memory is stored. The memory may store instructions that, when executed by the processor 202, can cause the surgical computing system 120 to perform one or more methods discussed herein.

The robot control system 214 may comprise a controller or microcontroller configured to control one or more robotic arm actuators and/or end effectors 226 to move robotic arms 114, 127, or 118 shown in FIG. 1. The robotic arms 114, 127, or 118 may cause movement and/or adjust the exoscope 106 and/or displays 132A-132B. Movement of the exoscope 106 may allow the field of view 130 associated with the exoscope 106 to center, and/or focus towards, a desired location in the surgical site 128 selected by a surgeon 112 or other operator during a surgical session. The desired location may be selected by identifying, among a plurality of labeled regions of the field of view, a labeled region corresponding to the desired location. Also or alternatively, the robot control system 214 may be configured to adjust one or more mechanical aspects of the exoscope 106 via camera adjustors 228, for example, to enable the exoscope 106 to zoom in or zoom out, adjust contrast or brightness, etc. The displays 132A-134B may output image data and/or video data stream captured by the exoscope 106, and which may be segmented and then augmented or rendered by the surgical computing system 120.

For example, the image segmentation module 210 may comprise a software, program, and/or computer executable instructions that cause the processor 202 to segment a received image data captured from the exoscope 106 into a plurality of regions. The segmentation may rely on one or more trained machine learning models 212 to assist in recognizing regions. For example, the exoscope 106 may be configured to capture at least one high-resolution image or video stream of the surgical field. This image data may be passed to the machine learning pipeline of the trained machine learning models 212 for processing.

According to an embodiment, the image data may undergo preprocessing steps such as resizing, normalization, noise reduction, and color correction. These steps may enhance the image quality and ensure that it matches the input specifications of the trained machine learning models 212 (e.g., consistent size, color depth, etc.). Subsequently, in accordance with some implementations, convolutional neural networks (CNNs) may be used for image segmentation due to their spatial hierarchies. Specific architectures like U-Net, Mask R-CNN, or DeepLab may be selected because they are highly effective for pixel-level image segmentation. The trained machine learning models 212 may determine which pixels belong to which anatomical structures using forward passes through the CNN layers. For example, a received image may undergo convolution, pooling, and activation operations, and feature maps may be generated at each layer of the CNN. Intermediate layers of the CNN may detect features like edges, textures, and shapes. The final layer may produce a pixel-wise probability distribution over classes using softmax or sigmoid activation, depending on whether it is a multi-class or binary segmentation task. A segmentation mask may be generated, where each pixel may be assigned a label corresponding to a specific region (e.g., different types of tissues, organs, etc.). This can be in the form of a color-coded overlay that maps directly onto the original image. Post-processing techniques, such as morphological operations or conditional random fields (CRFs), refine the segmentation mask, making boundaries smoother and reducing noise. Thereafter, the segmented areas may be mapped to create navigable regions, allowing the surgeon to distinguish between different parts of the anatomy. Each region may be labeled and defined based on clinical requirements. In an alternate embodiment, if the exoscope 106 supports depth perception (stereo imaging), the segmented regions may be combined into a 3D reconstruction, enabling even more precise navigation. The labeled regions may be rendered on the displays 132A-134B, overlaid on the live feed. Interactive tools may be provided, allowing the surgeon to focus on, magnify, or hide specific regions as needed. In one aspect, the image segmentation module 210 performs low-latency processing to ensure the segmented image aligns in real-time with the surgeon's movements. In dynamic surgeries where anatomical positions change, the trained machine learning models 212 may reprocess incoming frames obtained from the exoscope 106 continuously, providing updated segmentation to enable the surgeon to navigate within a constantly changing environment. Calibration between the exoscope 106 and the trained machine learning models 212 may be performed to ensure segmented regions align accurately with physical positions. Further, the segmentation output from the image segmentation module 210 may be validated by the surgeon to verify accuracy. Feedback from these validations may be used to retrain or refine the trained machine learning models 212.

In additional aspects, the trained machine learning models 212 may have been trained using training data comprising a plurality of reference image data (reference domain) having a plurality of recognized regions associated with the reference image data (reference range). The association formed between the reference domain and the reference range may be used to train the machine learning models (e.g., via deep learning), which may be relied on to segment image data received from the exoscope 106 (e.g., in real time or near real time).

For example, the trained machine learning models 212 may be trained for a variety of tasks such as object detection, segmentation, classification, and instance detection. During a medical procedure, a surgeon may refer to segmentation masks with a string of words in order to specify where she would like to navigate to such as “move to center of green mask” or “move to suction cannula.” The voice intent recognition module 218 may be configured to receive the surgeon's commands and translate them to a set of predefined definitions. A first portion of the command may indicate the location in the segmentation mask such as “left”, “center”, and “bottom. The second portion of the command may indicate the class label and/or the color of the mask such as “green” or “suction cannula.” The commands may also be translated by voice intent recognition module 218 to a set of predefined class labels and/or colors. As a result, the trained machine learning models 212 may determine which segmentation mask the surgeon would like to move to and where in a specific segmentation mask she would like to move to. Since it has been determined where the surgeon would like to move to in the image's coordinate system and the robot arms' coordinate system may be determined through e.g., a robot calibration process, the surgical computing system 120 may calculate the necessary joint speeds or transform for the robot arms to reposition the exoscope 106.

The augmentation may involve labeling the segmented regions. For example, the image augmentation module 224 may comprise a software, program, and/or computer executable instructions that cause the processor 202 to generate labels 208 to overlay or otherwise render the plurality of regions of the image data 206. The labels 208 may comprise different colors to color code the plurality of regions, for example, by rendering each region of the image data 206 to have a specific respective color. Also or alternatively, the labels 208 may comprise text to be overlaid on each of the plurality of regions, in order to provide easy identification for the region (e.g., Region A, Region B, Region 1, Region 2, Region A1, Region A2, etc.).

The localizer 216 may comprise hardware and/or software configured to detect pose of the exoscope 106, the computing system 120, the surgical site 128, and/or the surgical instrument 104 in a specified viewing space. The localizer 216 may supply this information to the computing system 120 responsive to requests for such information in periodic, real-time, or quasi-real-time fashion or at a constant rate. In some aspects, the localizer 216 may also be equipped with a localizer camera to also capture a field of view of the surgical site 128.

As the augmented image data is output as an image and/or video stream (augmented image and/or video) via the displays 132A-132B, the surgeon 112 may be prompted to select a labeled region for the exoscope 106 to center and/or shift focus to. The surgeon 112 may be enabled to select via a voice command based on voice captured by a microphone 126. The voice command may identify the region, for example, by a labeled color or identifying text. For example, the voice intent recognition module 218 may comprise an audio signal processing hardware and/or software configured to process the voice captured by the microphone 126 to detect sounds (e.g., words) that indicate and/or match one or more stored voice command templates 219. The voice command templates 219 may correspond to the labels 208 of the plurality of regions of the image data 206, and may be used by the voice intent recognition module 218 to recognize the voice command from the processed voice. The surgical computing system 120 may use the voice command to enable the robot control system 214 to move, position, and/or adjust the exoscope 106, via the robotic arm actuators and end effectors 226, to center or focus on a location of the surgical site 128 corresponding to the selected labeled region. In one embodiment, the surgical computing system 120 may create an error vector between a center pixel of screen and a target pixel on an image that a surgeon would like to move to. This error vector may be used to send a camera velocity to the surgical computing system 120 for moving the exoscope 106 to an identified location. In another embodiment, assuming the exoscope 106 has been calibrated, the surgical computing system 120 may determine the 3D location of a corresponding segmentation mask. Upon determining the 3D pose of the segmentation mask, the surgical computing system 120 may compute the desired destination for the exoscope 106 in the form of a transform with respect to the segmented mask. The segmentation-to-camera transform may then be translated to robot joint positions by using inverse kinematics.

In some embodiments, the surgeon may be enabled to select a desired region for the exoscope 106 to center or focus towards via a tapping gesture on a surgical instrument 104 while the surgical instrument 132 is touching or otherwise pointing towards a location of the surgical site 128 corresponding to the desired region. Also or alternatively, the tapping gesture may involve the surgical instrument 132 touching, pressing, clicking, or otherwise pointing towards a desired labeled region in the augmented image, for example, on a user interface 222 (e.g., a graphical user interface) or on the display 132A-132B. The instrument control module 220 may comprise hardware or software configured to detect the tapping gesture (e.g., via tap sensors on the instrument 132) and the region being pointed or touched (e.g., by tracking the instrument via image data received by the exoscope 106 and/or the localizer 216). The instrument control module 220 may thus be further configured to generate a control signal enabling the robot control system 214 to move, position, and/or adjust the exoscope 106, via the robotic arm actuators and end effectors 226, to center or focus on a location of the surgical site 128 corresponding to the selected labeled region.

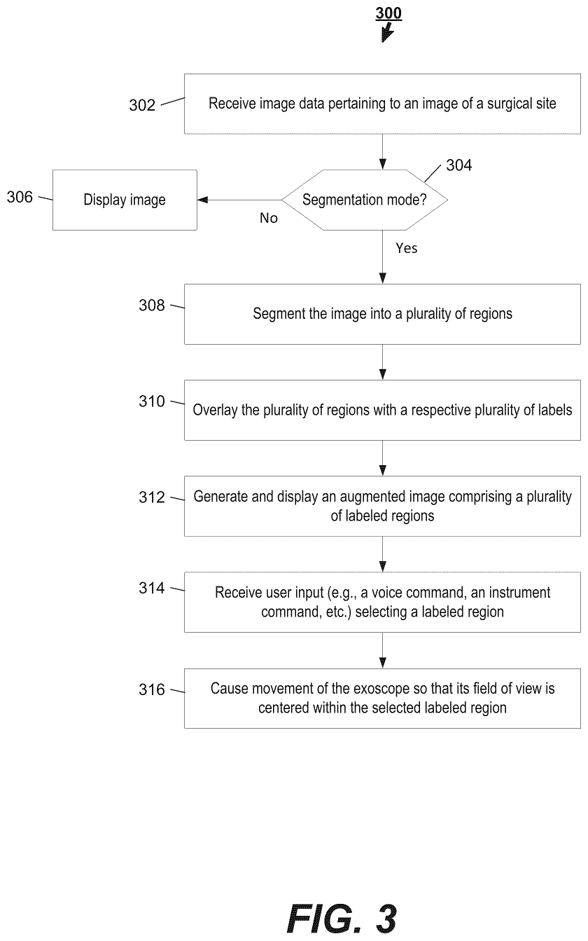

FIG. 3 is a block diagram of an example method 300 for performing on-demand deep learning based segmentation, in accordance with a non-limiting embodiment of the present disclosure. One or more steps of method 300 may be performed by the processor 202 of the surgical computing system 120 based on computer executable or machine readable instructions stored in the memory 204.

Method 300 may begin with the computing system 120 receiving image data pertaining to an image of the surgical site 128 (block 302). For example, image data captured by the exoscope 106 may be sent (e.g., transmitted via wired or wireless connections) to the computing system 120, where the processor 202 may receive the image data in real or near-time. The image data may comprise a digital representation of the analog image captured by the exoscope 106 in the form of pixels.

At block 304, the computing system 120 may determine whether segmentation mode is enabled. The segmentation mode may enable image navigation by labeling and augmenting image and/or video data stream captured by the exoscope 106, thus allowing the surgeon to select one or more regions of the image and automatically cause the exoscope 106 to center and/or focus on the selected region according to the systems and methods described herein. For example, a surgeon or operator of the surgical computing system 120 may toggle the segmentation mode on or off depending on their preference. If the segmentation mode is not enabled (e.g., the segmentation mode is turned off or deactivated), the computing system may thus display the current image 306 captured by the exoscope 106 (via displays 132A-132B). However, if segmentation mode is enabled, the computing system may proceed with steps described in subsequent blocks for segmenting and augmenting the image.

Thus, at block 308, the computing system 120 may segment the image corresponding to received image data into a plurality of regions (e.g., via the image segmentation module 210 using one or more trained machine learning models 212). The computing system 120 may apply one or more selected image-segmentation algorithms to identify navigable regions for the surgeon to select. For example, the computing system 120 may segment the image using semantic segmentation, instance segmentation, or panoptic segmentation. In some aspects, segmentation may occur by clustering regions of the image based on threshold intensity values for pixels (thresholding), using seed points of the image for growing regions based on similarity criteria (region growing), edge detection, clustering, watershed segmentation, or active contour detection.

In one aspect, the trained machine learning models 212 may be configured to learn a set of robust image features that encapsulate an image and subsequently use these image features to recover image segmentation masks that are as close as possible to the ground truth labels.

In at least one embodiment, the received image data may be segmented using deep learning models 212 (e.g., neural networks), which may be stored in the imaged segmentation module 210. For example, the computing system 120 may apply a series of a plurality of convolutional layers to the image data, based on set of filters. Each filter may comprise a matrix of weights that is convolved with the image data to produce a feature map. The feature map may represent a feature that the deep learning model (e.g., convolutional neural network model) may have been trained to detect. For example, the feature map may be used to detect edges, lines, and/or shapes for regions of images captured by the exoscope 106. In some aspects, the feature map may be obtained based on the training of a plurality of reference images having known (e.g., labeled) features (e.g., identified regions) within the images. In some aspects, the output of the convolution layers may be applied to an activation layer to introduce non-linearity. In some aspects, the output of the activation layer may be applied to a pooling layer for downsampling and/or reducing dimensionality (e.g., to make the segmentation computationally efficient). The output of the convolutional layers and/or the pooling layer may be applied to one or more fully connected layers to identify a plurality of regions (e.g., segments) of the image based on the inputted image data.

At block 310, the computing system may overlay the plurality of regions with a respective plurality of labels (e.g., via image augmentation module 214). The labels may comprise different colors for color coding the plurality of regions, for example, by rendering each region of the image data 206 to have a specific respective color. Also or alternatively, the labels 208 may comprise text to be overlaid on each of the plurality of regions, in order to provide easy identification for the region (e.g., Region A, Region B, Region 1, Region 2, Region A1, Region A2, etc.). For example, after the first frame, the segmentation masks may persist between frames (green mask on a suction cannula in frame 1 should continue to label the same suction cannula with a green mask).

At block 312, the computing system may generate and display an augmented image comprising a plurality of labeled regions. For example, the computing system may cause one or more of the displays 132A-132B to output the augmented image for the surgeon to view in real-time or near real-time.

The augmented image may prompt the surgeon or other viewer to select a labeled region of the augmented image in order to refocus and/or center the field of view of the exoscope 106. For example, the computing system may receive user input selecting a labeled region (block 314). The user input may comprise, for example, a voice command or an instrument command. For example, a voice command may be captured by a microphone 126. The voice command (“Blue,” “Blue Region,” “Region A,” “A,” etc.) may identify the region, for example, by a labeled color or identifying text. In an embodiment, the voice intent recognition module 218 of the computing system 120 may process the voice captured by the microphone 126 to detect sounds (e.g., words) that indicate and/or match one or more stored voice command templates 219. As discussed, the voice command templates 219 may correspond to the labels 208 of the plurality of regions of the image data 206. The voice intent recognition module 218 can thus use the voice command templates 219 to recognize the voice command from the processed voice as corresponding to a selected labeled region. The surgical computing system 120 may use the voice command to enable the robot control system 214 to move, position, and/or adjust the exoscope 106, via the robotic arm actuators and end effectors 226, to center or focus on a location of the surgical site 128 corresponding to the selected labeled region.

As used herein, an instrument command may comprise a functionality whereby the surgeon may be able to select a labeled region using an instrument or surgical tool. For example, the surgeon may generate a command signal by a tapping gesture on a surgical instrument while the surgical instrument is pointed towards a labeled region of the plurality of labeled region. The tapping gesture may be received and transmitted to the computing system 120 via a tap sensor. Also or alternatively, the tapping gesture and the pointing of the surgical instrument towards a labeled region intended for selection may be detected from image data generated via the exoscope 106.

At block 316, the computing system 120 may cause movement of the exoscope 106, so that its field of view is centered within the selected labeled region. For example, the computing system 120 may send electronic signals to one or more of the robotic arm actuators and end effectors 226 or camera adjustors 228 to move, position, and/or adjust the exoscope 106 to center or focus on a location of the surgical site 128 corresponding to the selected labeled region.

In some embodiments, the new field of view resulting from the new position of the exoscope 106 may cause the resulting image to be segmented into a plurality of new labeled regions, repeating one or more of the previously described steps of method 300.

In some embodiments, the computing system 120 may also generate a new plurality of regions within an existing region, for example, if a surgeon causes (e.g., via voice command) to focus (e.g., zoom in) within the existing region. The existing region may thus be further segmented based on methods described herein.

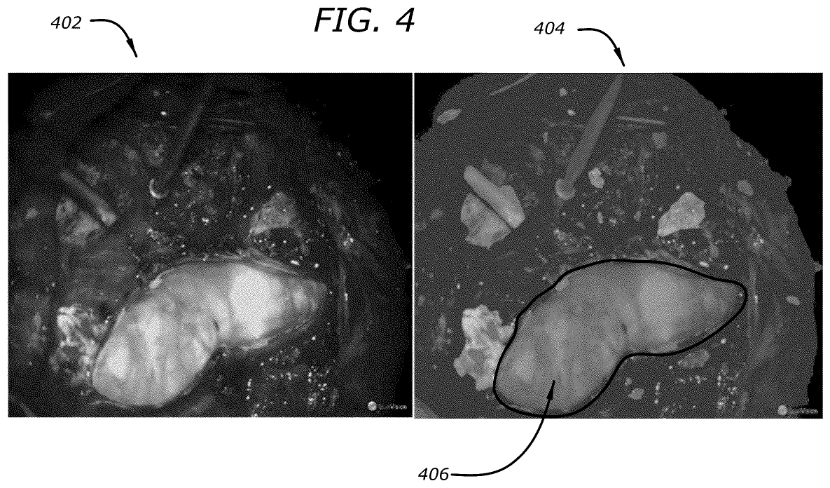

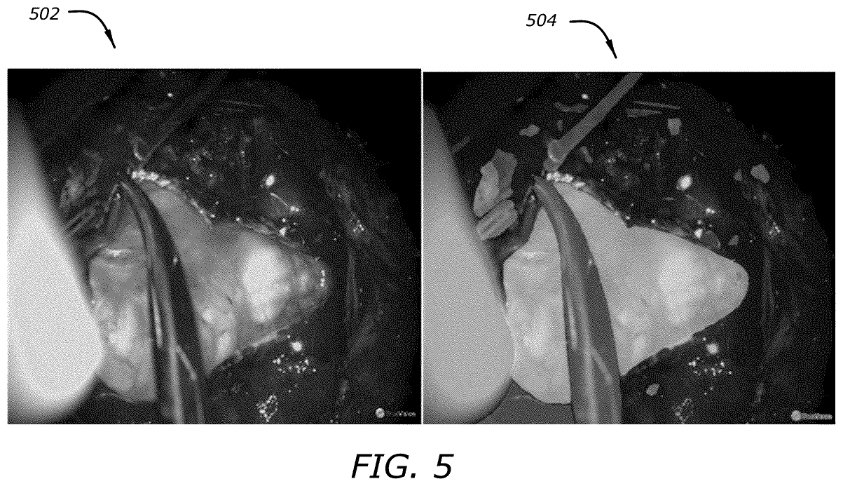

FIGS. 4 and 5 are image illustrations showing on-demand deep learning based segmentation, in accordance with a non-limiting embodiment of the present disclosure. For example, in FIG. 4, image 402 is an image captured by the exoscope 106 of a field of view comprising an anatomic site. The image 402 is not segmented yet, and thus is not easy for a surgeon or other viewer to be able to appropriately identify a region for the exoscope 106 to center and/or refocus on. However, image 404 is an augmented image output by one or more displays after the image 402 is processed and labeled according to methods presented herein. As shown in image 404, the augmented image includes a plurality of regions distinguished from one another based on color, which functions as a label. The surgeon can thus select a labeled region (e.g., region 406), for example, via a voice command identifying the color (e.g., “pink,” “pink region,” etc. to represent region 406), and cause the exoscope 106 to refocus and/or center its field of view to the pink region according to methods presented herein. FIG. 5 shows another set of images 502 and 504 illustrating the transformation from image captured by the exoscope 106 (image 502) to an augmented image output by one or more displays (image 504). As the surgical site captured by the exoscope 106 is different from that captured in FIG. 4, the labeled regions are thus different. Like the augmented image in FIG. 4, image 504 of FIG. 5 enables the surgeon to select a labeled region to cause the exoscope 106 to refocus and/or center its field of view on the selected labeled region.

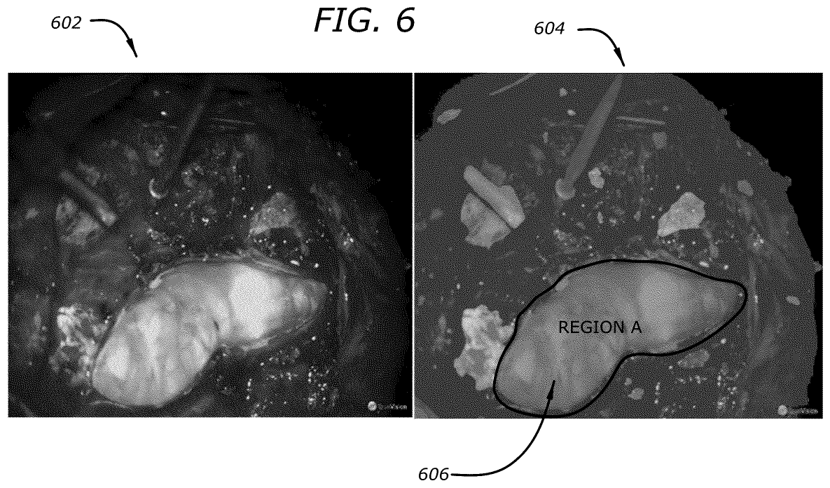

In another embodiment, FIG. 6 illustrates yet another set of images 602 and 604 transforming from an image captured by the exoscope 106 (image 602) to an augmented image output by one or more displays (image 604). Image 604 is an augmented image output by one or more displays after the image 602 is processed and labeled according to methods presented herein. As shown in image 604, the augmented image includes at least one identified and textually labelled region (e.g., Region A 606). The surgeon can thus select a labeled region (e.g., Region A606), for example, via a voice command, and cause the exoscope 106 to refocus and/or center its field of view to the labelled region according to methods presented herein.

It will be appreciated that each of the systems, structures, methods and procedures described herein may be implemented using one or more computer programs or components. These programs and components may be provided as a series of computer instructions on any conventional computer-readable medium, including random access memory (“RAM”), read only memory (“ROM”), flash memory, magnetic or optical disks, optical memory, or other storage media, and combinations and derivatives thereof. The instructions may be configured to be executed by a processor, which when executing the series of computer instructions performs or facilitates the performance of all or part of the disclosed methods and procedures.

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims. Moreover, consistent with current U.S. law, it should be appreciated that 35 U.S.C. 112 (f) or pre-AIA 35 U.S.C. 112, paragraph 6 is not intended to be invoked unless the terms “means” or “step” are explicitly recited in the claims. Accordingly, the claims are not meant to be limited to the corresponding structure, material, or actions described in the specification or equivalents thereof.

What is claimed is:

1. A system for image navigation using on-demand deep learning based segmentation, the system comprising:
an exoscope configured to capture image data from a field of view;
an image segmentation module;
an intent recognition module to capture a user's intent;
one or more robotic arms configured to move the exoscope;
a processor; and
memory storing computer-executable instructions that, when executed by the processor, causes the system to:
receive, via the exoscope, image data relating to an image or a video stream of a surgical site;
generate, via the image segmentation module, an augmented image comprising a plurality of labeled regions overlaying the surgical site;
receive, via the intent recognition module, a voice command selecting a labeled region of the plurality of labeled regions; and
cause, via the one or more robotic arms, a movement of the exoscope so that the selected labeled region is within the field of view of the exoscope after the movement.

2. The system of claim 1, wherein the plurality of labeled regions comprises one or both of:
a plurality of color coded regions; or
a plurality of textually labeled regions.

3. The system of claim 1, wherein the one or more robotic arms include at least one of a pneumatic arm and a hydraulic arm.

4. The system of claim 1, further comprising:
an instrument control module configured to receive a command signal caused by a tapping gesture on a surgical instrument while the surgical instrument is pointed towards a labeled region of the plurality of labeled region.

5. The system of claim 4, wherein the instructions, when executed by the processor, further cause the system to:
receive, via the instrument control module, an instrument command selecting a second labeled region of the plurality of labeled regions; and
cause, via the one or more robotic arms, a second movement of the exoscope so that the second selected labeled region is within the field of view of the exoscope.

6. The system of claim 1, wherein the instructions, when executed by the processor, cause the system to generate the augmented image by:
segmenting, using a deep learning model, the image of the surgical site into a plurality of regions.

7. The system of claim 1, further comprising:
a display configured to output the augmented image.

8. The system of claim 6, wherein the instructions, when executed by the processor, further cause the system to:
train the deep learning model using training data comprising a plurality of reference image data having a plurality of recognized regions associated with the reference image data.

9. The system of claim 8, wherein the instructions, when executed by the processor, cause the system to:
employ deep learning methods to associate each pixel of the image with a corresponding anatomical structure.

10. The system of claim 6, wherein the instructions, when executed by the processor, cause the system to segment, using the deep learning model, the image of the surgical site into the plurality of regions by at least one of:
clustering regions of the image of the surgical site based at least upon one of threshold intensity values for pixels;
using seed points of the image for growing regions based on similarity criteria; and
applying edge detection, watershed segmentation, or active contour detection.

11. A method for image navigation using on-demand deep learning based segmentation, the method comprising:
receiving, by a computing system having one or more processors, via an exoscope, image data relating to an image or a video stream of a surgical site;
generating, via a segmentation module associated with the computing system, an augmented image comprising a plurality of labeled regions overlaying the surgical site;
displaying the augmented image;
receiving, by the computing system, a user input selecting a labeled region of the plurality of labeled regions; and
causing, via one or more robotic arms supporting the exoscope, movement of the exoscope so that the selected labeled region is within a field of view of the exoscope after the movement.

12. The method of claim 11, wherein the user input is one or both of:
a voice command selecting a labeled region of the plurality of labeled regions; or
a tapping gesture on a surgical instrument while the surgical instrument is pointed towards a labeled region of the plurality of labeled regions.

13. The method of claim 11, further comprising, prior to generating the augmented image:
receiving a first user input to initiate segmentation,
wherein the augmented image is generated responsive to the first user input.

14. The method of claim 11, further comprising:
segmenting, via the segmentation module using a deep learning model, the image of the surgical site into a plurality of regions.

15. The method of claim 11, further comprising:
displaying, via a display associated with the computing system, the augmented image.

16. The method of claim 14, further comprising:
training, by the computing system, the deep learning model using training data comprising a plurality of reference image data having a plurality of recognized regions associated with the reference image data.

17. The method of claim 16, further comprising:
employing deep learning methods to associate each pixel of the image with a corresponding anatomical structure.

18. The method of claim 14, wherein the segmenting, using the deep learning model, the image of the surgical site into the plurality of regions comprises at least one of:
clustering regions of the image of the surgical site based at least upon one of threshold intensity values for pixels;
using seed points of the image for growing regions based on similarity criteria; and
applying edge detection, watershed segmentation, or active contour detection.

19. The method of claim 11, wherein the plurality of labeled regions comprises one or both of:
a plurality of color coded regions; or
a plurality of textually labeled regions.

20. The method of claim 11, wherein the one or more robotic arms include at least one of a pneumatic arm and a hydraulic arm.
